(19) United States
(12) Patent Application Publication
(10) Pub. No.: US 2013/0231598 A1
(43) Pub. Date: Sep. 5, 2013

(54) Foot Plate for Securing an Orthosis

(71) Applicants: David M. Velarde, Knoxville, TN (US); Michael Harris, Knoxville, TN (US)

(72) Inventors: David M. Velarde, Knoxville, TN (US); Michael Harris, Knoxville, TN (US)

(21) Appl. No.: 13/603,566

(22) Filed: Sep. 5, 2012

Related U.S. Application Data
(60) Provisional application No. 61/606,704, filed on Mar. 5, 2012.
(60) Provisional application No. 61/638,870, filed on Apr. 26, 2012.

(51) Int. Cl. A61F 5/00

(52) U.S. Cl. 602/27

(57) ABSTRACT

Disclosed is a foot plate to aid in the securement of an orthosis to a limb of a wearer. The foot plate may include a bottom member to be disposed along part of an underside of the wearer's foot and two attachment members to extend upwardly along the lateral and medial sides of the wearer's ankle to facilitate attachment of an orthosis thereto. The present general inventive concept enables wearers to limit the movement of an orthosis along the wearer's limb while also providing increased comfort to the foot and ankle of the wearer.

### CROSS-REFERENCE TO RELATED APPLICATIONS

Provisional Patent Application Ser. No. 61/606,704, filed on Mar. 5, 2012 and Provisional Patent Application Ser. No. 61/638,870, filed on Apr. 26, 2012, the contents of which are both incorporated herein by reference.

### BACKGROUND

1. Field of Invention

The present inventive concept relates generally to a plate for securing an orthosis to the limb of a wearer, and more particularly to a foot plate to be partially disposed beneath a wearer's foot and to extend up opposing sides of the wearer's ankle in order to conform thereto and limit movement of the attached orthosis with respect to the wearer's leg.

2. Description of Related Art

Securing orthoses to body limbs is desirable for many applications. Frequently, orthoses are secured to limbs in order to support and/or correct the limb during movement. Indeed, the term “orthosis” is often used to refer specifically to a device that supports or corrects the function of a limb. Additionally, however, orthoses may be secured to a limb of a wearer in order to provide a means for supporting and/or carrying an article. Therefore, as used herein, the term “orthosis” will be understood to refer to any type of device, apparatus, or structure that may be secured to a limb of the body, including but not limited to, sleeves, plates, straps, bands, belts, wraps, rods, etc.

Numerous designs of carrying devices for securing tools and other articles to a body of a person are known in the art. For example, in the field of firearms and firearm accessories, it is known to provide a holster or other similar carrying device to secure a firearm or accessory to the body of a user, for example to facilitate hands-free carrying of the firearm or firearm accessory. The term “holster” is often used to refer specifically to a carrying device for securing a firearm, and in particular a handgun, to the body of a user. However, as used herein, the term “holster” will be understood to include a carrying device for securing any of a number of articles, such as for example firearms, ammunition, ammunition magazines, flashlights, knives, hand tools, medical tools, navigation devices, communication devices, aiming devices, personal protection devices, non-lethal weapons, handcuffs and other restraint devices, wallets, passports, etc. Furthermore, for convenience, the term “firearm” as used herein will be understood to include any of a number of articles, including but not limited to the above-discussed articles, which a user may desire to secure to the user's body.

Ankle holsters for securing a firearm to an ankle of a person are usually designed with an orthosis, such as a strap or belt, to wrap around the ankle or the lower leg of the person, often just above the protruding ankle bone. Firearms are relatively heavy items, and when secured in these types of ankle holsters, they have a tendency to sling around during the articulated movement of the lower leg, especially when the wearer is engaging in intense physical activity such as running, kicking, crawling, etc. Thus, unless the ankle holster and/or orthosis are secured tightly against the lower leg, shifting of the orthosis, the ankle holster, and/or the associated firearm may occur. Such shifting can result in compromised support of the ankle joint, impairing smooth articulated movement of the wearer's leg and leading to discomfort and/or injury to the wearer. Such shifting can also result in decreased accessibility of the item carried in the holster by the user. In the case of a firearm secured in the holster, it is important for fast access and drawing of the firearm that the holster remain at a fixed position in relation to the leg, so that when a hand reaches down to withdraw the firearm, the firearm is in the fixed position in relation to the leg as expected by the wearer. In the case of so-called “retention holsters,” in which the holster is made to fit a firearm snugly to establish a releasable frictional connection between the firearm and the holster, shifting of the holster along the length of the wearer's leg can impede, and even prevent, drawing of the firearm from the holster.

To discourage shifting of an orthosis in relation to the wearer's leg, it is customary to secure the corresponding orthosis very tightly around the leg (e.g., with bands or straps), often times so tightly that vascular circulation to the wearer's ankle and foot is constricted or restricted. Such tight securement of the orthosis can result in discomfort to the user, such as for example by allowing the orthosis, holster, and/or firearm to dig into the skin and flesh of the wearer's leg. Such discomfort can be significant, and can even result in physical injury, and is especially likely when the orthosis is worn for extended periods of time or through intense physical activity.

Similarly, limb-supportive and/or limb-corrective orthoses also encounter the problem of shifting in relation to the limb of the wearer. Even with limited activity, such as walking, orthoses may experience shifting in relation to the limb of a wearer. In these contexts, the shifting of the orthosis may result in discomfort, physical injury, and/or impairment of the orthosis.

In light of the above, there is a need in the art for a device that allows securement of an orthosis to the leg of a wearer such that movement of the orthosis is limited in relation to the wearer's leg. Furthermore, there is a need for a device which may receive an orthosis and secure it to the leg or ankle of a wearer and which limits movement of the orthosis along the length dimension of the wearer's leg, but which also allows for increased comfort to the wearer by conforming to the wearer's foot and ankle.

### BRIEF SUMMARY

In accordance with various example embodiments of the present general inventive concept, a foot plate for securing an orthosis to a limb of a wearer may include a bottom member shaped to extend beneath at least a portion of an underside of a foot, the bottom member defining a medial side and a lateral side; and first and second attachment members, the first attachment member being secured at a first end thereof to the medial side and the second attachment member being secured at a first end thereof to the lateral side, the first and second attachment members each having an opposite second end provided with an orthosis attachment means. The orthosis attachment means may include a hinge provided with a cooperating member capable of engaging an orthosis. In some embodiments, the foot plate is made of a durable plastic that is both flexible and soft so as to provide comfort to the wearer and accommodate both narrow and wide feet by being able to conform thereto.

In some embodiments, the bottom member, the attachment members, or both are an elastomeric and/or flexible material capable of conforming to the limb of the wearer. Each of the first and second attachment members may be integrally formed with the bottom member. The bottom member, the first and/or second attachment member, or any combination thereof, may include notches bounding the first ends of the first and second attachment members. The first and second attachment members may each also have a plurality of alternating ridges and grooves on an interior surface thereof, an exterior surface thereof, or both.

In some embodiments, the bottom member has a longitudinal dimension and the first and second attachment members are secured to the bottom member such that they are longitudinally offset from one another. The first and second attachment members may be longitudinally offset from one another by a distance substantially equal to one centimeter. The medial side attachment member may be forward of the lateral side attachment member.

In some embodiments, the first and second attachment members each define a length dimension, the length dimension of the medial side attachment member being greater than the length dimension of the lateral side attachment member. The difference in length dimensions may be substantially equal to one centimeter.

Additional aspects and advantages of the present general inventive concept will be set forth in part in the description which follows, and, in part, will be obvious from the description, or may be learned by practice of the present general inventive concept.

### BRIEF DESCRIPTION OF THE FIGURES

The following example embodiments are representative of example techniques and structures designed to carry out the objects of the present general inventive concept, but the present general inventive concept is not limited to these example embodiments. In the accompanying drawings and illustrations, the sizes and relative sizes, shapes, and qualities of lines, entities, and regions may be exaggerated for clarity. A wide variety of additional embodiments will be more readily understood and appreciated through the following detailed description of the example embodiments, with reference to the accompanying drawings in which:

FIG. 1A illustrates a top down view of an interior surface of an example embodiment foot plate in accordance with the present general inventive concept;

FIG. 1B illustrates a top down view of an exterior surface of the foot plate of FIG. 1A;

FIG. 2 illustrates a perspective view of the example embodiment foot plate of FIGS. 1A and 1B;

FIG. 3A illustrates a side profile view of the example embodiment foot plate of FIG. 2;

FIG. 3B illustrates a rear profile view of the example embodiment foot plate of FIG. 2; and

FIG. 4 illustrates an example embodiment foot plate engaging an orthosis, both being worn on the leg of a wearer.

### DETAILED DESCRIPTION

Reference will now be made to various example embodiments of the present general inventive concept, examples of which are illustrated in the accompanying drawings and illustrations. The example embodiments are described herein in order to explain the present general inventive concept by referring to the figures. The following detailed description is provided to assist the reader in gaining a comprehensive understanding of the methods, apparatuses, and/or systems described herein. Accordingly, various changes, modifications, and equivalents of the methods, apparatuses, and/or systems described herein will be suggested to those of ordinary skill in the art.

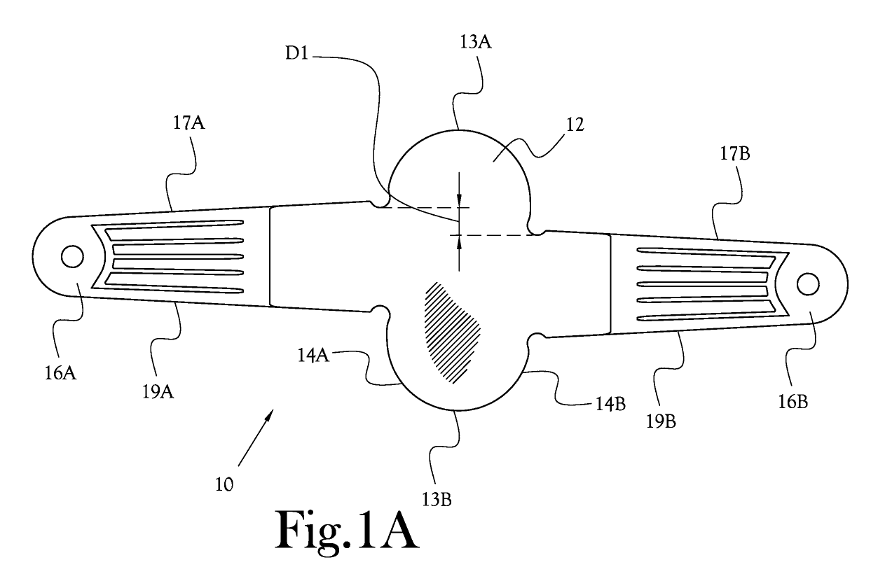

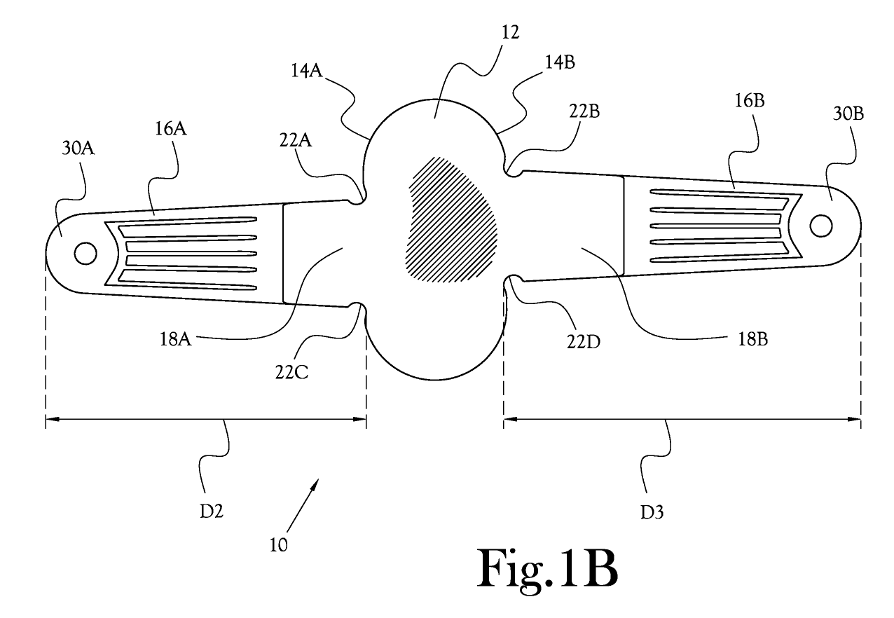

In accordance with various example embodiments of the present general inventive concept, a foot plate to aid in securing an orthosis to a limb of a wearer is provided. In some embodiments, the foot plate includes a bottom member generally sized and shaped to conform to and extend along a bottom portion of a wearer's foot beneath the wearer's ankle. First and second attachment members are provided to each of defined lateral and medial sides of the bottom member in order to facilitate the attachment of an orthosis thereto. FIGS. 1A-3B illustrate various views of an example embodiment foot plate for securing an orthosis thereto, in accordance with the present general inventive concept.

Referring to FIGS. 1A-3B, a foot plate for securing an orthosis 10 may generally include a substantially flat bottom member 12 defining a longitudinal dimension having rounded first and second ends 13A and 13B, a medial side 14A and a lateral side 14B. First and second attachment members 16A and 16B are secured at first ends 18A and 18B thereof to opposing medial 14A and lateral 14B sides of the bottom member 12 and are configured to extend upwardly from the bottom member 12 along the respective medial and lateral sides of the wearer's ankle. In the illustrated embodiment, respective first ends 18A and 18B of the first and second attachment members 16A and 16B are secured to the bottom member 12 by integral connection, and both the bottom member 12 and attachment members 16A and 16B are formed from a flexible and/or elastomeric material having sufficient tensile strength to support an orthosis. Examples of flexible material that may be used include, but are not limited to, rubber, polymers and/or plastics, cloth, leather, netting, etc. One of skill in the art will recognize, however, that the bottom member 12, the attachment members 16A and 16B, or both, may be formed from a rigid material, such as plastic, metal, wood, etc., without departing from the scope or spirit of the present general inventive concept.

A variety of options are available for coupling the first ends 18A and 18B of the first and second attachment members 16A and 16B to the respect lateral and medial sides of the bottom member 12. The presently illustrated example embodiment includes flexible attachment members 16A and 16B that have been integrally formed with the bottom member 12. One of skill in the art will understand that the attachment members 16A and 16B may also be independent entities coupled to the bottom member 12 through various means known in the art without departing from the scope or spirit of the present general inventive concept. Further, the first and second attachment members 16, 18 may be fixed in relation to the bottom member 12, such as in embodiments using a rigid material. It will also be understood that other suitable connections exist which may be used without departing from the spirit and scope of the present general inventive concept. For example, in one embodiment (not shown), respective first ends 18A and 18B of the first and second attachment members 16A and 16B are secured to respective medial and lateral sides 14A and 14B of the bottom member 12 by suitable hinges, such that the first and second attachment members 16A and 16B are rotatable about respective first ends 18A and 18B in relation to the bottom member 12.

The first and second attachment members 16A and 16B are provided to the bottom member 12 such that they may conform to the wearer's ankle. For instance, in some embodiments (not shown), the first and second attachment members 16A and 16B are symmetrically provided to the bottom member 12 on opposing medial and lateral sides 14A and 14B. In other embodiments, such as in the presently illustrated example embodiment foot plate 10, the first and second attachment members 16A and 16B asymmetrically provided to the bottom member 12 such that they are longitudinally offset from one another with respect to the longitudinal dimension of the bottom member 12. Stated differently, the first and second attachment members 16A and 16B each define first and second edges 17A and B and 19A and B, and the first edges 17A and 17B of the respective first and second attachment members 16A and 16B are offset from one another by a longitudinal offset distance D1 with respect to the longitudinal dimension of the bottom member 12. In some embodiments, the longitudinal offset distance D1 is substantially equal to one centimeter in order to accommodate the ankle bones of the wearer and maximize the comfort experienced by the wearer.

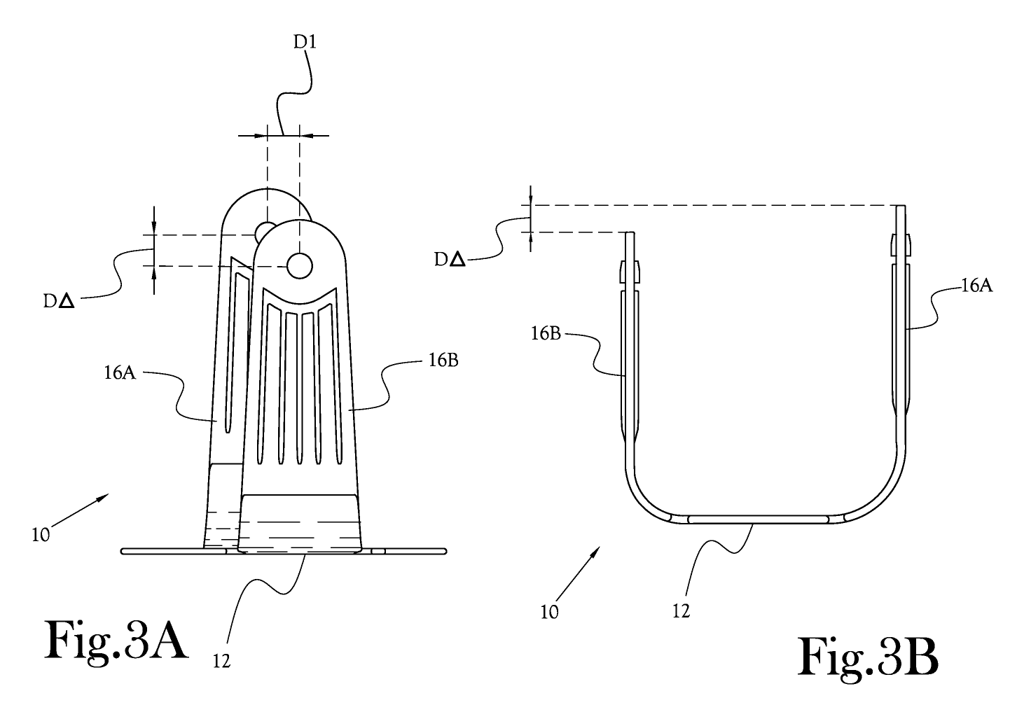

In some embodiments (not shown) the first and second attachment members 16A and 16B are equal in length. In other embodiments, such as the example embodiment illustrated in FIGS. 1A-3B, the first and second attachment members 16A and 16B are of differing lengths. Stated differently, one of the first and second attachment members 16A or 16B defines a longer length dimension D2 than the length dimension D3 defined by the other attachment member 16B or 16A. For example, in the illustrated example embodiment, the medial side attachment member 16A is approximately one centimeter longer than the lateral side attachment member 16B, as illustrated at DA in FIGS. 3A and 3B.

Still referring to FIGS. 1A-3B, the illustrated example embodiment foot plate 10 conforms to the right foot and ankle of a wearer. One of skill in the art will recognize that the wearer's medial malleolus may be disposed forward of and higher than the wearer's lateral malleolus. Thus, the presently illustrated example embodiment foot plate 10 is designed to accommodate this arrangement by its longitudinal offset distance D1 and the difference in length dimensions DA of the attachment members 16A and 16B. Because the first attachment member 16A provided to the medial side 14A of the bottom member 12 is disposed forward of the second attachment member 16B provided to the lateral side 14B of the bottom member 12, the foot plate 10 may conform to the wearer's ankle having a medial malleolus forward of the lateral malleolus. Further, because the medial side attachment member 16A is longer than the lateral side attachment member 16B, the foot plate 10 may conform to the wearer's ankle having a medial malleolus superior to their lateral malleolus. Stated differently, medial side attachment member 16A may be positioned to extend substantially over the wearer's leading and superior medial malleolus, while the lateral side attachment member 16B may be positioned to extend substantially over the wearer's following and inferior lateral malleolus. It will be understood that a corresponding left foot embodiment would reverse the above-described arrangement. One of skill in the art will also recognize that any and all arrangements, dimensions, and/or orientations discussed herein, including the lengths of the respective first and second attachment members, as well as their positions relative to one another and the bottom member 12, are merely example embodiments of the present general inventive concept. Accordingly, the disclosed arrangements, dimensions, and/or orientations may be modified without departing from the scope or spirit of the present general inventive concept.

Still referring to the example embodiments illustrated in FIGS. 1-3B, and specifically to FIG. 1B, notches 22A-D have been provided to the bottom member 12 on opposing sides of the first ends 18A and 18B of each attachment member 16A and 16B to further enable the foot plate 10 to conform to the wearer's ankle and foot. Stated differently, the first and second attachment members 16A and 16B are each bounded by notches 22A-D provided to the bottom member 12, thereby allowing the first and second attachment members 16A and 16B to conform to and extend up the wearer's ankle while also permitting the bottom member 12 to conform to the sole of the wearer's foot. It will be noted that while the presently illustrated example embodiment includes arcuate notches 22A-D, other shapes may be utilized without departing from the scope or spirit of the present general inventive concept. For instance, in some embodiments, the notches include a length dimension disposed substantially parallel to the first and second attachment members, the length dimension being substantially equal to 0.5 centimeters.

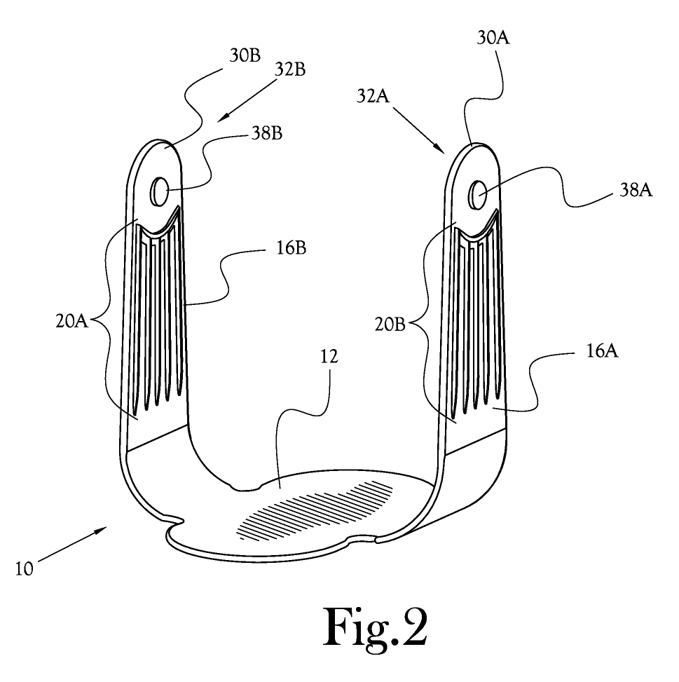

The foot plate 10 may include an interior surface (illustrated in FIG. 1A) to face the foot/ankle of the wearer and an opposite, exterior surface (illustrated in FIG. 1B). Either, neither, or both surfaces of each attachment member 16A and 16B may be provided with a ridges and grooves arrangement. For instance, in the example embodiment illustrated in FIG. 2, both surfaces of each attachment member 16A and 16B are provided with a ridges and grooves arrangement 20A and 20B, respectively, disposed in parallel with each attachment member 16A and 16B. The ridges and grooves arrangements 20A and 20B include a plurality of alternating ridges and grooves to maintain the flexible nature of, while also providing strength to, the attachment members 16A and 16B, and to discourage the attachment members 16A and 16B from flaring out away from the wearer's ankle during use. It should be noted that while the currently illustrated example embodiment includes ridges and grooves disposed in parallel with each attachment member 16A and 16B, other arrangements, including but not limited to perpendicularly disposed ridges and grooves, may be utilized without departing from the scope or spirit of the present general inventive concept.

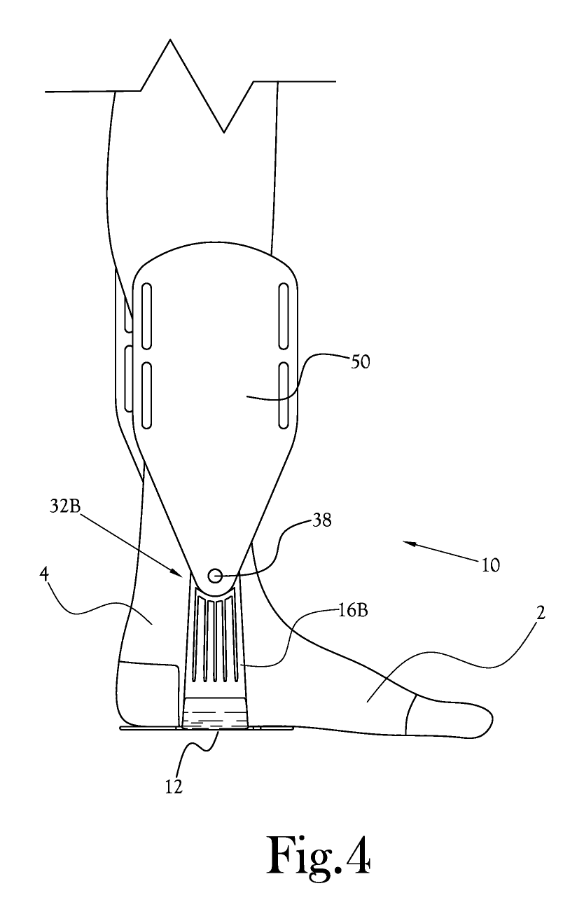

Each of the respective second ends 30A and 30B of the first and second attachment members 16A and 16B are provided with an orthosis attachment means such that they may be removably coupled to an orthosis. FIG. 2 illustrates example embodiment orthosis attachment means in the form of suitable hinges 32A and 32B. Each hinge 32A and 32B includes a cooperating member 38A and 38B in the form of a protrusion (illustrated) or a through-opening (not illustrated). In either case, the cooperating members 38A and 38B cooperatively engage an attachment means provided to an orthosis for securing the orthosis to the foot plate 10 and the leg of a wearer, as illustrated in FIG. 4. One of skill in the art will recognize, however, that the present general inventive concept is not limited to the specific orthosis attachment means provided herein (i.e., hinges). Accordingly, other orthosis attachment means may be used, including but not limited to, other types of suitable hinges, hook and loop fasteners, adhesives, snaps, buttons, clips, or the like, without departing from the scope or spirit of the present general inventive concept.

FIG. 4 illustrates the example embodiment foot plate 10 from FIGS. 1-3B rotatably coupled to an orthosis 50 on the right foot 2 and ankle 4 of a wearer. As illustrated, the bottom member 12 is disposed along part of the underside of the wearer's foot 2, with the lateral side attachment member 16B extending upwardly along the wearer's ankle 4. The hinge 32B, positioned substantially over the wearer's lateral malleolus, is engaging the lower part of the orthosis 50 to achieve a rotatable attachment thereto. Specifically, cooperating member 38 is cooperatively engaged with an attachment means provided to the orthosis 50, and is disposed through a through-opening provided thereto.

In the illustrated example embodiment, the orthosis 50 is a plurality of plates extending up the leg 6 of the wearer. One of skill in the art will recognize that the illustrated orthosis 50 may be a limb-supportive or limb-corrective orthosis, or may be further provided with an article carrying means, such as a holster, to accommodate the carrying of an article proximate the wearer's ankle 4.

In some unillustrated example embodiments, the position of attachment between the first and second attachment members 16A and 16B and the orthosis 50 is adjustable. For instance, each attachment member 16A and 16B may include a plurality of orthosis attachment means such that the selection of a particular orthosis attachment means modifies the position of attachment, such as with respect to the longitudinal dimension of the attachment member 16A and 16B. In some embodiments, two hinges are provided to each attachment member 16A and 16B, spaced apart along the longitudinal dimension of each. Alternatively, each hinge may include a plurality of cooperating members similarly spaced along each attachment member 16A and 16B. Thus, in these embodiments, the orthosis 50 may be selectively engaged with ether hinge/cooperating member to achieve the desired fit and comfort level for the wearer.

From the foregoing discussion, it will be recognized that a foot plate, in accordance with various example embodiments of the present general inventive concept, provides numerous advantages over the prior art. For instance, the foot plate 10 is adapted to engage a wearer's limb to receive and secure an orthosis 50 thereto such that the movement of the orthosis 50 is limited with respect to the length of the wearer's limb. In some embodiments, the foot plate 10 includes a bottom member 12 to conform to part of the underside of a wearer's foot 2, and two attachment members 16A and 16B extending upwardly along the lateral and medial sides of a wearer's ankle 4. The attachment members 16A and 16B may be bounded by notches 22A-D in the bottom member 12 to further enable the foot plate 10 to conform to the wearer's foot 2 and ankle 4. In some embodiments, the attachment members 16A and 16B are offset from one another with respect to the longitudinal dimension of the bottom member 12 to accommodate the wearer's medial malleolus being forward of the wearer's lateral malleolus. In some embodiments, the attachment member 16A extending along the medial side of the wearer's ankle 4 is longer than the attachment member 16B extending along the lateral side of the wearer's ankle 4 to accommodate the wearer's medial malleolus being superior to the wearer's lateral malleolus. Orthosis attachment means are also provided to the ends of each attachment member 16A and 16B to facilitate the removable coupling of an orthosis 50 thereto.

Numerous variations, modifications, and additional embodiments are possible, and accordingly, all such variations, modifications, and embodiments are to be regarded as being within the spirit and scope of the present general inventive concept. For example, regardless of the content of any portion of this application, unless clearly specified to the contrary, there is no requirement for the inclusion in any claim herein or of any application claiming priority hereto of any particular described or illustrated activity or element, any particular sequence of such activities, or any particular interrelationship of such elements. Moreover, any activity can be repeated, any activity can be performed by multiple entities, and/or any element can be duplicated.

While the present general inventive concept has been illustrated by description of several example embodiments, it is not the intention of the applicant to restrict or in any way limit the scope of the inventive concept to such descriptions and illustrations. Instead, the descriptions, drawings, and claims herein are to be regarded as illustrative in nature, and not as restrictive, and additional embodiments will readily appear to those skilled in the art upon reading the above description and drawings.

1. A foot plate for securing an orthosis to a limb of a wearer, the foot plate comprising:
a bottom member shaped to extend beneath at least a portion of an underside of a foot, the bottom member defining a medial side and a lateral side; and
first and second attachment members, the first attachment member being secured at a first end thereof to the medial side and the second attachment member being secured at a first end thereof to the lateral side, the first and second attachment members each having an opposite second end provided with an orthosis attachment means.

2. The foot plate of claim 1, wherein the bottom member, the attachment members, or both are an elastomeric material, and whereby the elastomeric material is capable of conforming to the limb of the wearer.

3. The foot plate of claim 1, wherein the bottom member, the attachment members, or both are a flexible material, and whereby the flexible material is capable of conforming to the limb of the wearer.

4. The foot plate of claim 1, wherein each of the first and second attachment members are integrally formed with the bottom member.

5. The foot plate of claim 4, wherein the bottom member, the first and/or second attachment members, or any combination thereof, include notches bounding the first ends of the first and second attachment members.

6. The foot plate of claim 1, wherein the first and/or second attachment members have a plurality of alternating ridges and grooves provided on an interior surface thereof, an exterior surface thereof, or both.

7. The foot plate of claim 1, wherein the bottom member has a longitudinal dimension, the first and second attachment members being secured to the bottom member such that they are longitudinally offset from one another.

8. The foot plate of claim 7, wherein the first and second attachment members are longitudinally offset from one another by a distance substantially equal to one centimeter.

9. The foot plate of claim 7, wherein the medial side attachment member is forward of the lateral side attachment member.

10. The foot plate of claim 1, wherein the first and second attachment members each define a length dimension, the length dimension of the medial side attachment member being greater than the length dimension of the lateral side attachment member.

11. The foot plate of claim 10, wherein the difference in length dimensions is substantially equal to one centimeter.

12. The foot plate of claim 1, wherein the orthosis attachment means includes a hinge provided with a cooperating member capable of engaging an orthosis.
